(19) United States
(12) Patent Application Publication
(10) Pub. No.: US 2026/0212972 A1
(43) Pub. Date: Jul. 23, 2026

(54) VERIFICATION MANAGEMENT DEVICE, VERIFICATION MANAGEMENT SYSTEM, AND VERIFICATION MANAGEMENT METHOD

(71) Applicant: George A. Bertalan, Rocky River, OH (US)

(72) Inventor: George A. Bertalan, Rocky River, OH (US)

(21) Appl. No.: 19/031,979

(22) Filed: Jan. 18, 2025

(51) Int. Cl. G16H 10/60; G06Q 50/00; H04L 9/32

(52) U.S. Cl. CPC G16H 10/60; H04L 9/3263; G06Q 10/40

(57) ABSTRACT

A verification management device may be configured to receive, from a medical testing facility device via a communication network, a set of medical data characterizing a health status of a first user; collect, from a verified data source over the communication network, a set of reliability factor data for evaluating reliability of the first user with respect to the set of medical data; determine, by evaluating the set of reliability factor data with respect to a set of reliability conditions, a reliability level of the first user with respect to the set of medical data; and issue, to a set of authorized third parties over the communication network, a verified digital credential corresponding to the reliability level.

### FIELD

The present disclosure generally relates to verification management. More particularly, the present disclosure relates to medical data verification for facilitating safety and accountability in an online dating context.

### BACKGROUND

Online dating services allow users to connect with potential partners by providing platforms to interact, communicate, and establish relationships based on shared interests, preferences, and characteristics. These services often include profile-based matching and messaging tools, facilitating interactions that can lead to platonic or romantic relationships. Over the years, online dating has become increasingly popular due to its convenience and accessibility.

However, as online dating continues to grow, significant challenges have emerged in ensuring safety, authenticity, and accountability within these platforms. One prominent issue is the prevalence of fraudulent profiles, catfishing, and deceptive behaviors, which undermine trust among users. Moreover, public health concerns, particularly those related to sexually transmitted diseases (STDs), add another layer of complexity to modern dating systems. Without mechanisms for screening or verifying health credentials, these platforms may inadvertently contribute to the spread of STDs, posing risks to individual users and public health at large.

In view of these challenges, there is a need for a secure, reliable system that provides independent verification of both personal health credentials and identity information without compromising privacy. Such a system would empower users with trust and accountability while fostering safer and more authentic interactions in online dating environments.

### SUMMARY

Aspects of the disclosure relate to a verification management device, system, and method for facilitating safety and accountability in an online dating context. The verification management device may be communicably connected to a medical testing facility device via a communication network such as the internet. The medical testing facility may include a clinic or hospital that offers medical testing services. The verification management device may receive a set of medical data characterizing a health status of a first user from the medical testing facility device via the communication network. Upon receiving the set of medical data, the verification management device may collect a set of reliability factor data for evaluating reliability of the first user with respect to the set of medical data. The set of reliability factor data may be collected from one or more verified data sources including a user device associated with the first user, the medical testing facility device, a set of dating service devices associated with different dating services, and a distributed ledger. The verification management device may evaluate the set of reliability factor data with respect to a set of reliability conditions to determine a reliability level of the first user with respect to the set of medical data. The verification management device may issue a verified digital credential corresponding to the reliability level to a set of authorized first parties including one or more dating platforms or services. The verified digital credential may be used to demonstrate reliability of the first user with respect to the set of medical data. For example, when used in an online dating context, the verified digital credential may be represented as a badge in the profile of the first user indicating that medical test results for the first user have been independently verified.

Aspects of the present disclosure relate to a verification management device including: a processor; and a memory connected to the processor and including a computer readable program, wherein: the verification management device is communicably connected, via a communication network, to at least a medical testing facility device associated with a medical testing facility, and the computer readable program, when executed on the processor, causes the processor to: receive, from the medical testing facility device via the communication network, a set of medical data characterizing a health status of a first user; collect, from a verified data source over the communication network, a set of reliability factor data for evaluating reliability of the first user with respect to the set of medical data; determine, by evaluating the set of reliability factor data with respect to a set of reliability conditions, a reliability level of the first user with respect to the set of medical data; and issue, to a set of authorized third parties over the communication network, a verified digital credential corresponding to the reliability level.

According to an embodiment of any paragraph(s) of this summary, determining the reliability level of the first user with respect to the set of medical data further includes: calculating, based on the set of reliability factor data, a reliability score for the first user; determining, by comparing the reliability score with a first threshold value; a relative relationship between the reliability score and the first threshold value; and assigning, from among a plurality of candidate reliability levels, the reliability level to the set of medical data based on the relative relationship between the reliability score and the first threshold value.

According to an embodiment of any paragraph(s) of this summary, the set of reliability factor data includes one or more reliability factors selected from the group consisting of: a medical testing frequency that indicates how frequently the first user receives medical testing; a medical testing date that indicates when a medical test was conducted; a number of matches of the first user on one or more dating platforms; an activity level of the first user on one or more dating platforms; and travel information indicating locations traveled to by the first user.

According to an embodiment of any paragraph(s) of this summary, calculating, based on the set of reliability factor data, the reliability score for the first user further includes: assigning a weighting value to each reliability factor of the set of reliability factor data; and computing the reliability score using a mathematical formula that defines a relationship between the one or more reliability factors and the weighting values assigned to each reliability factor.

According to an embodiment of any paragraph(s) of this summary, the verification management device is communicably connected via the communication network to a set of dating service devices each associated with a different dating service; and the verified data source includes one or more of a user device associated with the first user, the medical testing facility device, the dating service device, and a distributed ledger.

According to an embodiment of any paragraph(s) of this summary, the set of authorized third parties includes the set of dating service devices.

According to an embodiment of any paragraph(s) of this summary, collecting, from a verified data source over the communication network, the set of reliability factor data for evaluating reliability of the first user with respect to the set of medical data further includes: collecting, from a first dating service device of the set of dating service devices that is associated with a first dating service, a first number of matches of the first user on the first dating service; calculating, based on the first number of matches of the first user on the first dating service and a first baseline number of matches for users on the first dating service, a first normalized number of matches of the first user on the first dating service; collecting, from a second dating service device of the set of dating service devices that is associated with a second dating service, a second number of matches of the first user on the second dating service; calculating, based on the second number of matches of the first user on the second dating service and a second baseline number of matches for users on the second dating service, a second normalized number of matches of the first user on the second dating service; and calculating, based on the first normalized number of matches and the second normalized number of matches, a composite number of matches for the first user across the first dating service and the second dating service for use in calculating the reliability score for the first user with respect to the set of medical data.

According to an embodiment of any paragraph(s) of this summary, collecting, from a verified data source over the communication network, the set of reliability factor data for evaluating reliability of the first user with respect to the set of medical data further includes: collecting, from a first dating service device of the set of dating service devices that is associated with a first dating service, a first activity level of the first user on the first dating service; calculating, based on the first activity level of the first user on the first dating service and a first baseline activity level for users on the first dating service, a first normalized activity level of the first user on the first dating service; collecting, from a second dating service device of the set of dating service devices that is associated with a second dating service, a second activity level of the first user on the second dating service; calculating, based on the second activity level of the first user on the second dating service and a second baseline activity level for users on the second dating service, a second normalized activity level of the first user on the second dating service; and calculating, based on the first normalized activity level and the second normalized activity level, a composite activity level for the first user across the first dating service and the second dating service for use in calculating the reliability score for the first user with respect to the set of medical data.

According to an embodiment of any paragraph(s) of this summary, the activity level includes one or more of a number of logins to a dating service by the first user in a predetermined time period or an amount of time spent using a dating service by the first user in a predetermined time period.

According to an embodiment of any paragraph(s) of this summary, the set of medical data indicates test results of medical testing of the first user for sexually transmitted diseases.

According to an embodiment of any paragraph(s) of this summary, the processor is further configured to: collect, from the verified data source, a second set of reliability factor data; determine, by evaluating the second set of reliability factor data with respect to the set of reliability conditions, a second reliability level of the first user with respect to the set of medical data; and issue, to the set of authorized third parties, an updated verified digital credential corresponding to the second reliability level.

According to an embodiment of any paragraph(s) of this summary, the processor is further configured to: provide a recommendation to the first patient for increasing the reliability level.

According to an embodiment of any paragraph(s) of this summary, the processor is further configured to: identify, based on the set of reliability factor data, at least one improvement factor for improving the reliability level of the first user; determine at least one suggested activity which affects the at least one improvement factor to increase the reliability level; and provide a recommendation of the at least one suggested activity to the first user.

According to an embodiment of any paragraph(s) of this summary, the set of medical data is encrypted by the medical testing facility device and transmitted through a secure channel of the communication network, the set of medical data is associated with a first authenticated ID, and the processor is further configured to: receive, from a user device via the communication network, a user account creation request associated with a second authenticated ID of the first user; create a user account for the first user based on the user account creation request; decrypt the set of medical data received from the medical testing facility device; verify an identify of the first user by comparing the first authenticated ID associated with the set of medical data with the second authenticated ID associated with the user account creation request; and associate, in response to determining that the first authenticated ID associated with the medical data matches the second authenticated ID associated with the user account creation request, the set of medical data with the user account.

According to an embodiment of any paragraph(s) of this summary, the verified digital credential is issued to the set of authorized third parties using Public Key Infrastructure.

According to an embodiment of any paragraph(s) of this summary, issuing, to a set of authorized third parties over the communication network, the verified digital credential corresponding to the reliability level further includes: generating a key pair including a public key and a private key; generating the verified digital credential based at least on an authenticated ID for the first user and the reliability level determined for the first user; digitally signing the verified digital credential by generating a hash corresponding to the verified digital credential and encrypting the hash using the private key; and transmitting the verified digital credential to the set of authorized third parties for validation using the public key.

According to an embodiment of any paragraph(s) of this summary, issuing, to a set of authorized third parties over the communication network, the verified digital credential corresponding to the reliability level further includes: recording the verified digital credential to a distributed ledger.

According to an embodiment of any paragraph(s) of this summary, receiving, from the medical testing facility device via the communication network, the set of medical data characterizing a health status of the first user further includes: retrieving, using a transaction identifier associated with the first user, the set of medical data from a distributed ledger to which the set of medical data has been recorded by the medical testing facility device.

Aspects of the present disclosure relate to a verification management system including: a verification management device; a user device associated with a first user; a distributed ledger; and a medical testing facility device associated with a medical testing facility, wherein the verification management device, the user device, the distributed ledger and the medical testing facility device are communicably connected via a communication network, the verification management device including: a processor; and a memory connected to the processor and including a computer readable program that when executed by the processor, causes the processor to: receive, from the user device via the communication network, a user account creation request associated with user registration information, the user registration information including a first authenticated ID for the first user; receive, from the medical testing facility device via the communication network, a set of medical data indicating medical test results for a first patient, the set of medical data associated with a second authenticated ID for the first patient; create a user account for the first user based on the user account creation request; verify, by comparing the second authenticated ID associated with the set of medical data for the first patient with the first authenticated ID included in the user registration information for the first user, that the first patient corresponds to the first user; collect, from a verified data source over the communication network, a set of reliability factor data for evaluating reliability of the first user with respect to the set of medical data; store, in the distributed ledger, the set of medical data and the set of reliability factor data in association with the first authenticated ID for the first user; calculate, based on the set of reliability factor data, a reliability score for the first user; determine, by comparing the reliability score with a first threshold value; a relative relationship between the reliability score and the first threshold value; assign, from among a plurality of candidate reliability levels, the reliability level to the set of medical data based on the relative relationship between the reliability score and the first threshold value; and issue, to a set of authorized third parties over the communication network, a verified digital credential corresponding to the reliability level.

Aspects of the present disclosure relate to a verification management method for a verification management device, the verification management device including: a processor; and a memory connected to the processor and including a computer readable program, wherein: the verification management device is communicably connected, via a communication network, to at least a medical testing facility device associated with a medical testing facility, and the computer readable program, when executed on the processor, causes the processor to perform a verification management method including: receiving, from the medical testing facility device via the communication network, a set of medical data characterizing a health status of a first user; collecting, from a verified data source over the communication network, a set of reliability factor data for evaluating reliability of the first user with respect to the set of medical data; determining, by evaluating the set of reliability factor data with respect to a set of reliability conditions, a reliability level of the first user with respect to the set of medical data; and issuing, to a set of authorized third parties over the communication network, a verified digital credential corresponding to the reliability level.

The above summary is not intended to describe each illustrated embodiment or every implementation of the present disclosure.

### BRIEF DESCRIPTION OF THE DRAWINGS

The accompanying drawings, which are incorporated in and constitute a part of the specification, illustrate various example systems, methods, and so on, that illustrate various example embodiments of aspects of the invention. It will be appreciated that the illustrated element boundaries (e.g., boxes, groups of boxes, or other shapes) in the figures represent one example of the boundaries. One of ordinary skill in the art will appreciate that one element may be designed as multiple elements or that multiple elements may be designed as one element. An element shown as an internal component of another element may be implemented as an external component and vice versa. Furthermore, elements may not be drawn to scale.

FIG. 1 illustrates an example computing architecture for executing the embodiments of the present disclosure.

FIG. 2 is a diagram illustrating an example hardware configuration of a verification management system according to the embodiments of the present disclosure.

FIG. 3 is a diagram illustrating a verification management method according to the embodiments of the present disclosure.

FIG. 4 is a diagram illustrating an example of the flow of a reliability factor aggregation method according to the embodiments of the present disclosure.

FIG. 5 is a diagram illustrating an example of an activity suggestion method according to the embodiments of the present disclosure.

FIG. 6 is a diagram illustrating a particular example of the flow of the verification management system according to the embodiments of the present disclosure.

### DETAILED DESCRIPTION

Hereinafter, embodiments of the present disclosure will be described with reference to the accompanying drawings. It should be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure. Additionally, it should be noted in the following description that the same reference numerals in different embodiments denote the same or similar features.

As described herein, as online dating services have grown in popularity, challenges have emerged in ensuring safety, authenticity, and accountability within these platforms. One prominent issue is the prevalence of fraudulent profiles, catfishing, and deceptive behaviors, which undermine trust among users. Moreover, public health concerns, particularly those related to sexually transmitted diseases (STDs), add another layer of complexity to modern dating systems. Accordingly, aspects of the present disclosure relate to a verification management technique for providing independent verification of personal health credentials and identity information without compromising privacy. For example, the verification management technique may verify medical data (e.g., STD test results) provided by medical facilities for users of online dating services, and issue verified digital credentials to the dating services to demonstrate the veracity of the user's identity and health status. For example, when used in an online dating context, the verified digital credential may be represented as a badge in the profile of a user indicating that the identity and medical test results for the user have been independently verified. In this way, it becomes possible to reduce the prevalence of fraudulent profiles, catfishing, and deceptive behaviors while fostering safer and more authentic interactions in online dating environments.

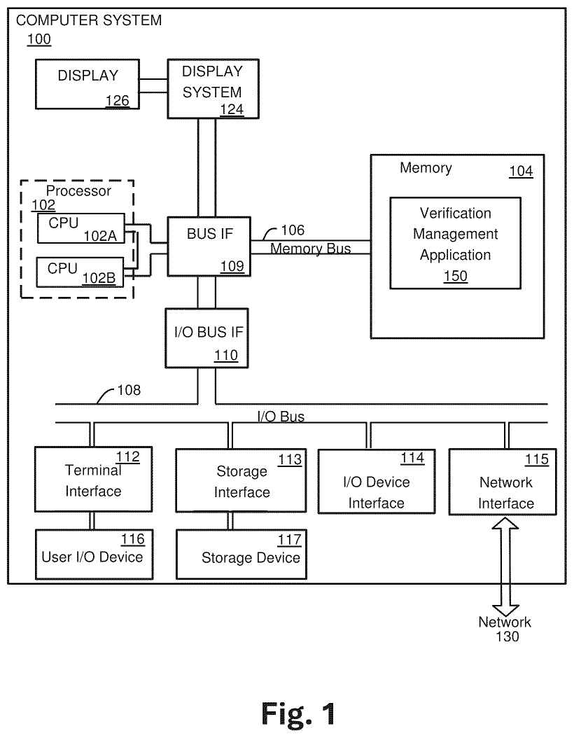

Turning now to the Figures, FIG. 1 depicts a high-level block diagram of a computer system 100 for implementing various embodiments of the present disclosure, according to embodiments. For example, the verification management device as explained below can be implemented by the computer system 100. The mechanisms and apparatus of the various embodiments disclosed herein apply equally to any appropriate computing system. The major components of the computer system 100 include one or more processors 102, a memory 104, a terminal interface 112, a storage interface 113, an I/O (Input/Output) device interface 114, and a network interface 115, all of which are communicatively coupled, directly or indirectly, for inter-component communication via a memory bus 106, an I/O bus 108, bus interface unit 109, and an I/O bus interface unit 110.

The computer system 100 may contain one or more general-purpose programmable central processing units (CPUs) 102A and 102B, herein generically referred to as the processor 102. In embodiments, the computer system 100 may contain multiple processors; however, in certain embodiments, the computer system 100 may alternatively be a single CPU system. Each processor 102 executes instructions stored in the memory 104 and may include one or more levels of on-board cache.

In embodiments, the memory 104 may include a random-access semiconductor memory, storage device, or storage medium (either volatile or non-volatile) for storing or encoding data and programs. In certain embodiments, the memory 104 represents the entire virtual memory of the computer system 100 and may also include the virtual memory of other computer systems coupled to the computer system 100 or connected via a network. The memory 104 can be conceptually viewed as a single monolithic entity, but in other embodiments the memory 104 is a more complex arrangement, such as a hierarchy of caches and other memory devices. For example, memory may exist in multiple levels of caches, and these caches may be further divided by function, so that one cache holds instructions while another holds non-instruction data, which is used by the processor or processors. Memory may be further distributed and associated with different CPUs or sets of CPUs, as is known in any of various so-called non-uniform memory access (NUMA) computer architectures.

The memory 104 may store all or a portion of the various programs, modules and data structures for processing data transfers as discussed herein. For instance, the memory 104 can store a verification management application 150. In embodiments, the verification management application 150 may include instructions or statements that execute on the processor 102 or instructions or statements that are interpreted by instructions or statements that execute on the processor 102 to carry out the functions as further described below.

In certain embodiments, the verification management application 150 is implemented in hardware via semiconductor devices, chips, logical gates, circuits, circuit cards, and/or other physical hardware devices in lieu of, or in addition to, a processor-based system. In embodiments, the verification management application 150 may include data in addition to instructions or statements. In certain embodiments, a camera, sensor, or other data input device (not shown) may be provided in direct communication with the bus interface unit 109, the processor 102, or other hardware of the computer system 100. In such a configuration, the need for the processor 102 to access the memory 104 and the verification management application 150 may be reduced.

The computer system 100 may include a bus interface unit 109 to handle communications among the processor 102, the memory 104, a display system 124, and the I/O bus interface unit 110. The I/O bus interface unit 110 may be coupled with the I/O bus 108 for transferring data to and from the various I/O units. The I/O bus interface unit 110 communicates with multiple I/O interface units 112, 113, 114, and 115, which are also known as I/O processors (IOPs) or I/O adapters (IOAs), through the I/O bus 108. The display system 124 may include a display controller, a display memory, or both. The display controller may provide video, audio, or both types of data to a display device 126. Further, the computer system 100 may include one or more sensors or other devices configured to collect and provide data to the processor 102.

As examples, the computer system 100 may include biometric sensors (e.g., to collect heart rate data, stress level data), environmental sensors (e.g., to collect humidity data, temperature data, pressure data), motion sensors (e.g., to collect acceleration data, movement data), or the like. Other types of sensors are also possible. The display memory may be a dedicated memory for buffering video data. The display system 124 may be coupled with a display device 126, such as a standalone display screen, computer monitor, television, or a tablet or handheld device display.

In one embodiment, the display device 126 may include one or more speakers for rendering audio. Alternatively, one or more speakers for rendering audio may be coupled with an I/O interface unit. In alternate embodiments, one or more of the functions provided by the display system 124 may be on board an integrated circuit that also includes the processor 102. In addition, one or more of the functions provided by the bus interface unit 109 may be on board an integrated circuit that also includes the processor 102.

The I/O interface units support communication with a variety of storage and I/O devices. For example, the terminal interface unit 112 supports the attachment of one or more user I/O devices 116, which may include user output devices (such as a video display device, speaker, and/or television set) and user input devices (such as a keyboard, mouse, keypad, touchpad, trackball, buttons, light pen, or other pointing device). A user may manipulate the user input devices using a user interface in order to provide input data and commands to the user I/O device 116 and the computer system 100 and may receive output data via the user output devices. For example, a user interface may be presented via the user I/O device 116, such as displayed on a display device, played via a speaker, or printed via a printer.

The storage interface 113 supports the attachment of one or more disk drives or direct access storage devices 117 (which are typically rotating magnetic disk drive storage devices, although they could alternatively be other storage devices, including arrays of disk drives configured to appear as a single large storage device to a host computer, or solid-state drives, such as flash memory). In some embodiments, the storage device 117 may be implemented via any type of secondary storage device. The contents of the memory 104, or any portion thereof, may be stored to and retrieved from the storage device 117 as needed. The I/O device interface 114 provides an interface to any of various other I/O devices or devices of other types, such as printers or fax machines. The network interface 115 provides one or more communication paths from the computer system 100 to other digital devices and computer systems; these communication paths may include, for example, one or more networks 130.

Although the computer system 100 shown in FIG. 1 illustrates a particular bus structure providing a direct communication path among the processors 102, the memory 104, the bus interface 109, the display system 124, and the I/O bus interface unit 110, in alternative embodiments the computer system 100 may include different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface unit 110 and the I/O bus 108 are shown as single respective units, the computer system 100 may, in fact, contain multiple I/O bus interface units 110 and/or multiple I/O buses 108. While multiple I/O interface units are shown which separate the I/O bus 108 from various communications paths running to the various I/O devices, in other embodiments, some or all of the I/O devices are connected directly to one or more system I/O buses.

In various embodiments, the computer system 100 is a multi-user mainframe computer system, a single-user system, or a server computer or similar device that has little or no direct user interface but receives requests from other computer systems (clients). In other embodiments, the computer system 100 may be implemented as a desktop computer, portable computer, laptop or notebook computer, tablet computer, pocket computer, telephone, smart phone, or any other suitable type of electronic device.

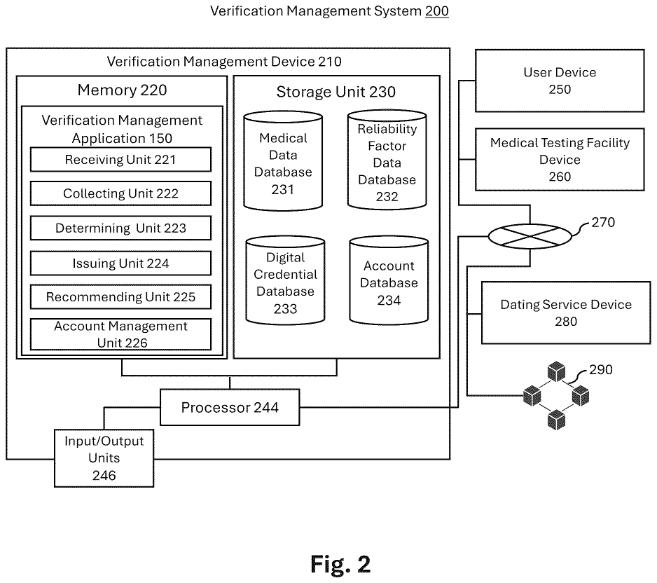

Next, with reference to FIG. 2, an example hardware configuration of a verification management system 200 according to the embodiments of the present disclosure will be described.

FIG. 2 is a diagram illustrating an example hardware configuration of a verification management system 200 according to the embodiments of the present disclosure. The verification management system 200 relates to a system configured to verify the reliability of a user with respect to a set of medical data. In embodiments, aspects of the functionality of the verification management system 200 may be provided as a service (e.g., subscription-based service) to users.

As used herein, “reliability” refers to the authenticity, validity, accuracy, veracity, or trustworthiness of an entity such as a human user or data. Additionally, as used herein, the phrase “reliability of the user with respect to the set of medical data” will be understood to refer to the degree of confidence that the medical data associated with the user accurately represents the current health status of the user. As described herein, the reliability of the user with respect to the set of medical data may be evaluated based on a set of reliability factor data characterizing a set of reliability factors.

Further, as used herein, “the set of medical data” refers to data characterizing the health status of a user based on a medical evaluation. In embodiments, the set of medical data may include information indicating the time and location that a user has undergone medical testing at a licensed medical facility such as a hospital or clinic but omit detailed results of the medical testing. In embodiments, the set of medical data may include detailed medical test results in addition to information indicating the time and location that the user has undergone medical testing. While the set of medical data may relate to medical test results to evaluate any aspect of a user's health, aspects of the disclosure relate to embodiments in which the set of medical data specifically relates to medical testing results indicating the presence or absence of sexually transmitted diseases (STDs). Accordingly, for convenience of explanation, aspects of the present disclosure will be described with reference to an example in which the set of medical data includes data relating to medical test results for STDs. As examples, STDs may include gonorrhea, chlamydia, syphilis, Human Immunodeficiency Virus (HIV), Human Papillomavirus (HPV), hepatitis B and C, herpes simplex virus, or the like.

As illustrated in FIG. 2, the verification management system 200 according to the embodiments of the present disclosure may include a verification management device 210, a user device 250, a medical testing facility device 260, a dating service device 280, and a distributed ledger 290. The verification management device 210 may be communicatively connected to the user device 250, the medical testing facility device 260, the dating service device 280, and the distributed ledger 290 via a communication network 270. Here, the communication network 270 may include a Local Area Network (LAN) connection, the Internet, a Wide Area Network (WAN) connection, a Metropolitan Area Network (MAN) connection, a Bluetooth (registered trademark) connection, or the like.

The verification management device 210 is an apparatus configured to facilitate verification of the reliability of a user with respect to a set of medical data. As illustrated in FIG. 2, the verification management device 210 primarily includes a memory 220, a storage unit 230, a processor 244, and an input/output unit 246. In embodiments, aspects of the verification management device 210 may be implemented using a computer system such as the computer system 100 illustrated in FIG. 1.

The memory 220 is a memory for storing a verification management application 150 for implementing the functions of the verification management technique according to the embodiments of the present disclosure. The verification management application 150 may be a computer-readable program. As illustrated in FIG. 2, the verification management application 150 may include a receiving unit 221, a collecting unit 222, a determining unit 223, an issuing unit 224, a recommending unit 225 and an account management unit 226. The receiving unit 221, collecting unit 222, determining unit 223, issuing unit 224, recommending unit 225 and account management unit 226 may be implemented as software modules constituting the verification management application 150.

The receiving unit 221 is a functional unit for receiving a set of medical data. The receiving unit 221 may receive the set of medical data from the medical testing facility device 260 described later. As described herein, the set of medical data may include information relating to the time and location (e.g., name and ID of medical facility) that a first user has undergone medical testing (e.g., STD testing), as well as detailed results of the medical testing. The receiving unit 221 may store the received set of medical data in the medical data database 231. As the detailed functionality of the receiving unit 221 will be provided later herein, a description thereof will be omitted here.

The collecting unit 222 is a functional unit for collecting a set of reliability factor data for evaluating reliability of the first user with respect to the set of medical data. The collecting unit 222 can collect the set of reliability factor data from a verified data source over the communication network 270. In embodiments, the verified data source can include one or more of the user device 250, the medical testing facility device 260, the dating service device 280, or the distributed ledger 290. The collecting unit 222 may store the collected set of reliability factor data in the reliability factor data database 232.

As used herein, the set of reliability factor data refers to a collection of data for evaluating reliability of the first user with respect to the set of medical data. The set of reliability factor data may include one or more reliability factors including a medical testing frequency that indicates how frequently the first user receives medical testing (e.g., STD testing frequency), a medical testing date that indicates when a medical test was conducted (e.g., how recent the latest medical test was), a number of matches of the first user on one or more dating platforms (e.g., number of other users with which a match has been approved), an activity level of the first user on one or more dating platforms, and travel information indicating locations traveled to by the first user.

The medical testing frequency may indicate a frequency at which the first user receives medical testing (e.g., STD testing). The medical testing frequency can be a weekly frequency (times/week), monthly frequency (times/month), or annual frequency (times/year). The medical testing date may indicate a date when the first user last received a particular medical test, or the duration of time from when the user last received a particular medical test. The number of matches on one or more dating platforms may indicate the number of other users that the first user has been matched with on one or more dating platforms. The activity level on one or more dating platforms may indicate the number of logins to one or more dating platforms within a predetermined time period (e.g., logins per day, week, month, or year), or the length of time that a user spends actively on one or more dating platforms (e.g., browsing for matches, messaging other users). The travel information may indicate information about a time when the first user traveled to a particular location and the time (e.g., date) of the travel. In embodiments, the medical testing frequency and/or the medical testing date may be collected from the medical testing facility device 260 and/or the distributed ledger 290. In embodiments, the number of matches on one or more dating platforms and/or the activity level on one or more dating platforms can be collected from the dating service device 280 and/or the distributed ledger 290. In embodiments, the travel information can be collected from the user device 250 and/or the distributed ledger 290. As the detailed functionality of the collecting unit 222 will be provided later herein, a description thereof will be omitted here.

The determining unit 223 is a functional unit for determining a reliability level of the first user with respect to the set of medical data. The determining unit 223 can determine the reliability level by evaluating the set of reliability factor data with respect to a set of reliability conditions. As used herein, the reliability level refers to a discrete rank or grade indicating the degree of reliability of the first user with respect to the set of medical data. In embodiments, the reliability level assigned to a particular set of reliability factor data may be selected from among a pre-determined number of candidate reliability levels. As an example, the reliability level may be selected from among pre-determined candidate reliability levels of “Platinum,” “Gold,” “Silver,” and “Bronze” where “Platinum” indicates a relatively high degree of reliability and “Bronze” indicates a relatively lower degree of reliability. Additionally, as used herein, the set of reliability conditions may refer to a set of rules, criteria, parameters, values, or relationships for assessing the degree of reliability of the first user with respect to the set of medical data based on the set of reliability factor data. In embodiments, the set of reliability conditions may include a formula or equation for calculating a reliability score for the first user with respect to the set of medical data based on the set of reliability factor data. As the detailed functionality of the determining unit 223 will be provided later herein, a description thereof will be omitted here.

The issuing unit 224 is a functional unit for issuing a verified digital credential corresponding to the reliability level determined by the determining unit. The issuing unit 224 may issue the verified digital credential to a set of authorized third parties over the communication network 250. As used herein, the verified digital credential refers to digital, cryptographically secured information indicating the relative reliability of the first user with respect to the set of medical data. The issuing unit 224 may store information regarding the issued verified digital credentials in the digital credential database 233. In embodiments, the verified digital credential may be protected using cryptographic techniques such as public-private key infrastructure (PKI) and stored in a secure format such as a digital wallet or the distributed ledger 290. For instance, in embodiments, the issuing unit 224 may generate the verified digital credential for the first user and digitally sign it with a private key to ensure the authenticity and integrity of the credential. This verified digital credential may be validated by an authorized third party using a public key associated with the verification management service 210. In embodiments, the verified digital credential may be generated and issued in accordance with the standards of the W3C Verifiable Credentials (VC) framework. As the detailed functionality of the issuing unit 223 will be provided later herein, a description thereof will be omitted here.

In embodiments, the set of authorized third parties may include one or more dating service devices 280. Once the verified digital credential has been issued by the issuing unit 224, the dating service device 280 may use the verified digital credential to reflect the reliability level of the first user with respect to the set of medical test data on a dating platform. For example, the dating service device 280 may represent the verified digital credential using a badge displayed in the profile of the first user on the dating platform that reflects the reliability level (e.g., Platinum, Gold, Silver, Bronze) of the user. In certain embodiments, the reliability level indicated by the verified digital credential may be used as a search criterion to facilitate matches between individuals with corresponding reliability levels.

In embodiments, the set of authorized third parties may include the user device 250. The verified digital credential may be stored in a secure digital wallet to allow a user of the user device 250 to selectively share the verified digital credential with other entities. In certain embodiments, the set of authorized third parties may include the distributed ledger 290, such that the verified digital credential can be recorded in the distributed ledger 290 in a permanent, immutable fashion.

The recommending unit 225 is a functional unit for providing a recommendation for increasing the reliability level of the first user with respect to the set of medical data. The recommending unit 229 may be configured to identify at least one improvement factor for improving the reliability level of the first user, determine at least one suggested activity which affects the at least one improvement factor to increase the reliability level, and provide a recommendation of the at least one suggested activity to the first user. As the detailed functionality of the recommending unit 223 will be provided later herein, a description thereof will be omitted here.

The account management unit 226 is a functional unit for creating and managing accounts for users of services offered by the verification management system 200. In embodiments, the account management unit 226 may receive a user account creation request from the user device 250 via the communication network 270 to create an account for a first user and create a user account for the first user. Information regarding the accounts created for users may be stored in the account database 234. As the detailed functionality of the account management unit 226 will be provided later herein, a description thereof will be omitted here.

The storage unit 230 is a unit for storing various data and information used in implementing the aspects of the present disclosure. The storage unit 230 may include a collection of hard disk drives, solid state drives, flash memory, cloud, storage, or the like. As illustrated in FIG. 2, the storage unit 230 may include a medical data database 231, a reliability factor data database 232, a digital credential database 233, and an account database 234.

The medical data database 231 is a database configured to store the set of medical data received by the receiving unit 221. In embodiments, the medical data database 231 may be formatted as a table including the contents of the medical data for the first user and an identifier for the account associated with the first user.

The reliability factor data database 232 is a database configured to store the set of reliability factor data collected by the collecting unit 222. In embodiments, the reliability factor data database 232 may be formatted as a table including the contents of the set of reliability factor data for the first user and an identifier for the account associated with the first user.

The digital credential database 233 is a database configured to store verified digital credentials issued by the issuing unit 228. In embodiments, the digital credential database 233 may include a digital wallet for securely storing the verified digital credentials. In embodiments, the digital credential database 233 may be formatted as a table including an identifier of the verified digital credentials for the first user and an identifier for the account associated with the first user.

The account database 234 is a database configured to store information regarding accounts created for users by the account management unit 226. In embodiments, the account database 234 may be formatted as a table including an identifier for the account associated with the user, passwords for the first user, authenticated IDs for the first user, and the like.

The processor 244 is a processing unit for executing processing instructions for the various software modules and functional units included in the verification management application 150 stored in the memory 220 and may substantially correspond to the processor 102 of the computer system 100 illustrated in FIG. 1.

The input/output units 246 are a collection of devices for facilitating communication between the verification management device 210 and external sources, such as an administrator of the verification management device 210, the user device 250, the medical testing facility device 260, the dating service device 280, and the distributed ledger 290 to which the verification management device 210 is communicably connected. In embodiments, the input/output units 246 may include a built-in display unit, speakers, network card, or the like for facilitating the input and output of information with respect to the verification management device 210.

The user device 250 is a device that can be used by a user of the services/functionality provided by the verification management device 210. In embodiments, the user device 250 may be used to send an account creation request to the verification management device 210 to initiate creation of a user account for a first user. In certain embodiments, the user device 250 may be used to provide a portion of the set of reliability factor data (e.g., travel information, passport number) to the verification management device 210 via the communication network 270. As examples, the user device 250 may be implemented using a personal computer, tablet computer, smart phone, or other computing device.

The medical testing facility device 260 is a device associated with a medical testing facility such as a clinic or hospital. In embodiments, the medical testing facility device 260 may be used to provide the set of medical data to the verification management device 210. For example, after a user (e.g., the first user) has undergone medical testing at the medical testing facility, a set of medical data indicating the details of the medical testing may be transmitted by the medical testing facility device 260 to the verification management device 210 via a secure channel of the communication network 270. As examples, the user device 250 may be implemented using a personal computer, server, tablet computer, smart phone, or other computing device.

The dating service device 280 is a device associated with a dating platform or service. In embodiments, the dating service device 280 may be used to provide a portion of the set of reliability factor data (e.g., number of matches, activity level) to the verification management device 210 via the communication network 270. Additionally, in embodiments, the verified digital credential issued by the issuing unit 224 may be transmitted to the dating service device 280 for use on an associated dating platform. As examples, the user device 250 may be implemented using a personal computer, server, tablet computer, smart phone, or other computing device.

The distributed ledger 290 (also referred to as a shared ledger, or distributed ledger technology) is a system whereby replicated, shared, and synchronized digital is geographically spread (distributed) across many sites, countries, or institutions. The distributed ledger may be implemented using a peer-to-peer (P2P) computer network and consensus algorithms such that the ledger is reliably replicated across distributed computer nodes (servers, clients, etc.). As an example, the distributed ledger 290 may be a blockchain implemented on a public or private network. In embodiments, the distributed ledger 290 may be used to record various types of data used to implement aspects of the present disclosure in a permanent, immutable format. For example, the set of medical data, the set of reliability factor data, the verified digital credentials, or user account information may be stored on the distributed ledger 290.

It will be understood that while an example of the verification management system 200 was described as including a verification management device 210, a user device 250, a medical testing facility device 260, a dating service device 280, and a distributed ledger 290, the verification management system 200 is not limited herein, and the configuration of the verification management system 200 may be suitably modified. Additionally, it will be understood that the verification management device 210 can be connected with a plurality of user devices 250, a plurality of medical testing facility devices 260, a plurality of dating service devices 280, and a plurality of distributed ledgers 290 to facilitate service scaling and provisioning for a plurality of users.

According to the verification management device 210 illustrated in FIG. 1, it is possible to provide a verification management technique capable of issuing a verified digital credential demonstrating the reliability of a user with respect to a set of medical data while protecting user privacy. This verified digital credential can be used in a dating platform to foster safer and more authentic interactions in online dating environments.

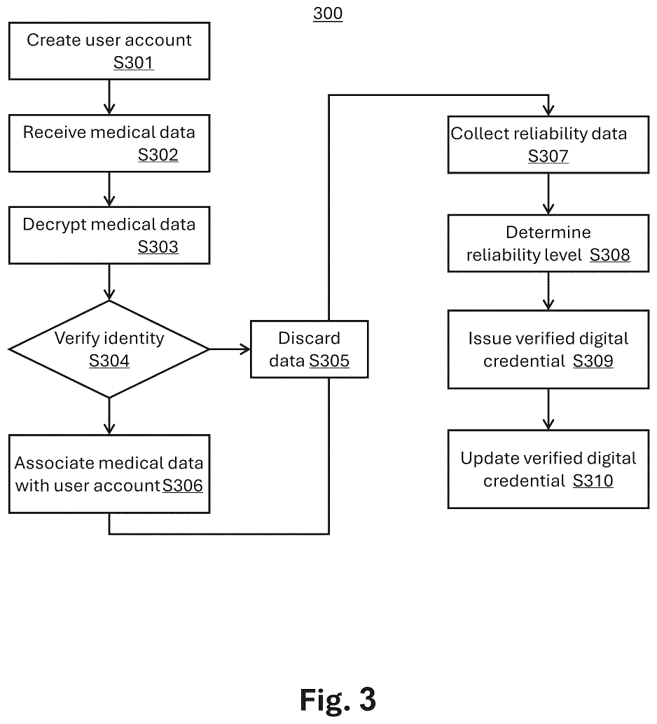

Next, with reference to FIG. 3, an example of a verification management method according to the embodiments of the present disclosure will be described.

FIG. 3 is a diagram illustrating an example of the flow of a verification management method 300 according to the embodiments of the present disclosure. The verification management method 300 relates to a method for verifying the reliability of a user with respect to a set of medical data. The verification management method 300 may be implemented by the components of the verification management system 200 described above with reference to FIG. 2.

At Step S301, the account management unit 226 may create a user account for a first user. Here, the user account refers to a digital identity established to provide the first user with access to services provided by the verification management system 200. In embodiments, the account management unit 226 may receive, from the user device 250, a user account creation request. The user account creation request may include user registration information necessary for account creation, such as a desired username, password, security information, and the like. In embodiments, the user registration information may include a second authenticated ID. The second authenticated ID may include an identity document for proving the first user's identity based on personal identification information such as full name, physical appearance, birth date, address, identification number, gender, citizenship, or the like. For example, the second authenticated ID may include a passport, driver's license, residence card, social security card, birth certificate, health insurance card, or the like. In response to receiving the user account creation request, the account management unit 226 may generate an entry in the account database 234 to store the information included in the user account creation request in association with a unique account identifier. In this way, a user account granting access to services provided by the verification management system 200 can be created for the first user.

In embodiments, the user registration information may include dating service account information identifying one or more dating service accounts used by the first user that the first user wishes to link with the verification management system 200 of the present disclosure. For example, the user registration information may include a username and password of a first dating service account on a first dating platform and a username and password of a second dating service account on a second dating platform. This dating service information may be used to link the user account created for the user in the verification management system 200 with the dating service accounts of the first user to facilitate provision of a verified digital credential.

Next, at Step S302, the receiving unit 221 may receive a set of medical data from the medical testing facility device 260. As described herein, the set of medical data may include data characterizing the health status of a first patient based on a medical evaluation. In embodiments, the set of medical data may include information indicating the time and location that the first patient has undergone medical testing (e.g., STD testing) at a licensed medical facility such as a hospital or clinic, and optionally detailed results of the medical testing. In embodiments, the set of medical data may include information regarding past medical testing performed for the first patient. The set of medical data may include a first authenticated ID for the first patient. The first authenticated ID may include an identity document for proving the first patient's identity based on personal identification information such as full name, physical appearance, birth date, address, identification number, gender, citizenship, or the like. For example, the first authenticated ID may include a passport, driver's license, residence card, social security card, birth certificate, health insurance card, or the like. In embodiments, the set of medical data may be encrypted by the medical testing facility using one or more encryption methods including but not limited to Transport Layer Security (TLS), Advanced Encryption Standard (AES), Public Key Infrastructure (PKI), Virtual Private Network (VPN), Secure File Transfer Protocol (SFTP), End-to-End Encryption (E2EE) or the like. The set of medical data may be transmitted directly from the medical testing facility to the verification management device 210 over a secure network connection.

In embodiments, the medical testing facility device 260 may record the set of medical data for the first user to a distributed ledger (e.g., the distributed ledger 290 illustrated in FIG. 2) in association with a particular transaction identifier associated with the first user. Subsequently, the receiving unit 221 may retrieve the set of medical data from the distributed ledger using the transaction identifier associated with the first user. In other embodiments, the receiving unit 221 may receive the set of medical data directly from the medical testing facility device 260 and subsequently store it on the distributed ledger in association with an authenticated ID for the first user. The set of medical data and the authenticated ID may be hashed and digitally signed (e.g., using the principles of PKI or the W3C Verified credentials framework) prior to recording to the distributed ledger to protect the privacy of the first user.

Next, at Step S303, the receiving unit 221 may decrypt the set of medical data received from the medical testing facility device 260 at Step S302. In embodiments, the receiving unit 221 may employ a decryption technique selected in accordance with the type of encryption used to encrypt the set of medical data by the medical testing facility device. For example, in the case that the set of medical data was encrypted in a hash using a public/private key according to a PKI configuration, the receiving unit 221 may use a corresponding private/public key to decode the hash and decrypt the set of medical data. The receiving unit 221 may store the decrypted set of medical data in the medical data database 231.

Next, at Step S304, the receiving unit 221 may verify that the first patient characterized by the set of medical data received from the medical testing facility in Step S302 corresponds to the first user for whom a user account was created in Step S301. More particularly, the receiving unit 221 may verify that the first patient corresponds to the first user by comparing the second authenticated ID associated with the set of medical data for the first patient with the first authenticated ID included in the user registration information for the first user. As used herein, verifying that the first patient corresponds to the first user refers to determining that the first patient matches the first user; that is, that the first patient and the first user are the same individual. For example, the receiving unit may verify that the first patient corresponds to the first user in the case that the name, physical appearance, birth date, address, identification number, or other personal information matches (or satisfies a predetermined similarity threshold) between the second authenticated ID and the first authenticated ID. The comparison of the first and second authenticated IDs may be performed using any suitable comparison or matching algorithm known to the those skilled in the art. In the case that the identity of the first patient associated with the set of medical data is verified to correspond to the identity of the first user associated with the registration information, the method proceeds to Step S306. In the case that the identity of the first patient associated with the set of medical data could not be verified to correspond to the identity of the first user associated with the registration information, the method proceeds to Step S305.

At Step S305, in the case that the identity of the first patient associated with the set of medical data could not be verified to correspond to the identity of the first user associated with the registration information, the receiving unit 221 discards the set of medical data received at Step S302. In this way, the privacy of the first patient can be facilitated.

At Step S306, in response to verifying that the identity of the first patient associated with the set of medical data corresponds to the identity of the first user associated with the registration information, the receiving unit 221 associates the set of medical data received at Step S302 with the user account created at Step S301. More particularly, the receiving unit 221 may modify the medical data database 231 and the user account database 234 to include a unique identifier for the set of medical data and a unique identifier for the user account stored in association with each other. In this way, the set of medical data and the user account can be linked together. In certain embodiments, the receiving unit 221 may record the unique identifier for the user account in association with the unique identifier for the set of medical data to the distributed ledger 290.

Next, at Step S307, the collecting unit 222 collects, from a verified data source over the communication network, a set of reliability factor data for evaluating reliability of the first user with respect to the set of medical data. As described herein, the set of reliability factor data refers to a collection of data for evaluating reliability of the first user with respect to the set of medical data. The verified data source may include one or more of the user device 250, the medical testing device 260, the dating service device 280, and the distributed ledger 290. In embodiments, collecting the set of reliability factor data may include querying the medical testing device 260, the dating service device 280, and the distributed ledger 290 for particular information related to the first user identified in Step S304.

Aspects of the present disclosure relate to the recognition that the reliability of the set of medical data, and therefore the reliability of the user associated with the set of medical data, may vary based on a variety of reliability factors including a medical testing frequency that indicates how frequently the first user receives medical testing (e.g., STD testing frequency), a medical testing date that indicates when a medical test was conducted (e.g., how recent the latest medical test was), a number of matches of the first user on one or more dating platforms (e.g., number of other users with which a match has been approved), an activity level of the first user on one or more dating platforms, and travel information indicating locations traveled to by the first user.

For example, with regard to a medical testing frequency, a user with a greater frequency of medical testing may be associated with a higher degree of reliability as compared to a user with a lower frequency of medical testing, because more frequent medical testing may reduce the chance of misdiagnosis and provide a more complete representation of the health status of the patient. Similarly, with regard to medical testing date, a set of medical data based on medical testing conducted several days ago may be considered to have a high degree of reliability, whereas a set of medical data based on medical testing conducted several weeks or months ago may be considered to have lower reliability, as it may no longer accurately reflect the current health of the patient. In the context of a dating platform, with regard to a number of matches, users with a lower number of matches may be considered to have a higher degree of reliability as they have a lower number of potential encounters and therefore a lower likelihood of exposure to communicable diseases (e.g., STDs). Additionally, with regard to activity level, users with a lower activity level (e.g., in terms of number of logins or time spent using a dating service) may be considered to have a higher degree of reliability as they are less engaged in dating activities and therefore have a lower likelihood of exposure to communicable diseases (e.g., STDs). Further, with regard to travel information, users who visited a country or region with high infection rates of a particular disease may be considered to have a lower degree of reliability as they had a higher likelihood of exposure to communicable diseases (e.g., STDs). It will be understood that the set of reliability factors are not limited herein, and a variety of other factors may be considered.

In embodiments, collecting the set of reliability data may include parsing the set of medical data using a natural language processing technique to extract the set of reliability factors. For example, the collecting unit 222 may parse the set of medical data using a natural language processing technique to extract a medical testing frequency that indicates how frequently the first user receives medical testing (e.g., STD testing frequency) and a medical testing date that indicates when a medical test was conducted from the set of medical data. As examples, the natural language processing techniques used to extract the reliability factors from the set of medical data may include named entity recognition, regular expression matching, dependency parsing, part-of-speech tagging, contextual language models adapted for medical texts, rule-based temporal tagging, information retrieval and contextual search, semantic role labeling, ontology-based techniques, or hybrid approaches that combine multiple techniques (e.g., named entity recognition for initial detection and rule-based methods for further refinement).

In embodiments, collecting the set of reliability data may include querying one or more dating service devices 280 associated with one or more dating platforms to retrieve a number of matches of the first user on the one or more dating platforms (e.g., number of other users with which a match has been approved) and the activity level of the first user on the one or more dating platforms. More particularly, the collecting unit 222 may query one or more dating services for sets of reliability data associated with the dating service account information included in the registration information provided by the first user for user account creation. In this way, the particular reliability data associated with the dating service accounts used by the first user may be ascertained and retrieved. In certain embodiments, as described later herein, the collecting unit 222 may normalize reliability factors such as the number of matches and the activity level from a plurality of dating platforms and aggregate them together to calculate a composite value that provides a representation of the aggregated reliability factors across the plurality of dating platforms.

In embodiments, collecting the set of reliability data may include querying the user device 250 to retrieve passport information for the first user that may be used to obtain a record indicating travel information for the first user.

In embodiments, collecting the set of reliability data may include accessing the distributed ledger 290 to obtain one or more reliability factors that have been committed to the distributed ledger. For example, the collecting unit 222 may access the distributed ledger 290 to obtain the medical testing frequency or medical testing data for the first user recorded by a medical testing facility, a number of matches or an activity level of the first user on one or more dating platforms recorded by a dating platform, or travel information (e.g., passport number) for the first user recorded by the first user.

Next, at Step S308, the determining unit 223 may determine a reliability level of the first user with respect to the set of medical data by evaluating the set of reliability factor data collected in Step S307 with respect to a set of reliability conditions. As described herein, the reliability level refers to a discrete rank or grade indicating the degree of reliability of the first user with respect to the set of medical data. In embodiments, the reliability level assigned to a particular set of reliability factor data may be selected from among a pre-determined number of candidate reliability levels. As an example, the reliability level may be selected from among pre-determined candidate reliability levels of “Platinum,” “Gold,” “Silver,” and “Bronze” where “Platinum” indicates a relatively high degree of reliability and “Bronze” indicates a relatively lower degree of reliability. Additionally, as used herein, the set of reliability conditions may refer to a set of rules, criteria, parameters, values, or relationships for assessing the degree of reliability of the first user with respect to the set of medical data based on the set of reliability factor data. In embodiments, the set of reliability conditions may include a formula or equation for calculating a reliability score for the first user with respect to the set of medical data based on the set of reliability factor data.

In embodiments, the determining unit 226 may determine the reliability level of the first user with respect to the set of medical data by calculating a reliability score for the first user based on the set of reliability factor data collected in Step S307, determining a relative relationship between the reliability score and a first threshold value by comparing the reliability score with the first threshold value, and assigning the reliability level from among a plurality of candidate reliability levels to the set of medical data based on the relative relationship between the reliability score and the first threshold value.

As used herein, the reliability score (RS) refers to a quantitative representation of the degree of reliability of the first user with respect to the set of medical data. In embodiments, the reliability score may be a real number calculated using a mathematical formula that defines a relationship between the reliability factors of the set of reliability factor data and weighting values assigned to each reliability factor. As an example, in certain embodiments, the reliability score may be computed using the following Equation 1:

$$RS = ( \frac{{w}_{T}}{T} ) + ( {w}_{F} \times F ) - ( {w}_{M} \times M ) - ( {w}_{A} \times A ) - ( {w}_{L} \times L ) \qquad Equation 1$$

In Equation 1, T refers to the time that has elapsed since the most recent medical testing date (e.g., in days), F refers to a medical testing frequency (e.g., number of tests in the last year), M refers to a number of matches in a predetermined time period, A refers to an activity level (e.g., number of logins or number of usage hours) on one or more dating platforms, and L refers to a risk factor user determined based on a user's travel history. The weight values W_{T}, W_{F}, W_{M}, W_{A}, and W_{L} can be assigned to the respective reliability factors to modify the impact a specific reliability factor has on the overall reliability score and may be determined by an administrator of the verification management system 200.

The calculated reliability score can be compared with one or more threshold values to determine a relative relationship between the reliability score and the one or more threshold values. Here, the one or more threshold values may define particular reliability scores that serve as boundaries between different reliability levels. The number of threshold values may be determined based on the desired number of reliability levels used for classifying the reliability of the user. For example, the reliability score may be compared with respect to one, two, or three or more threshold values. Additionally, the threshold values may be set to fixed values by an administrator of the verification management system 200 or set to variables that change based on characteristics of the first user. For example, the threshold values may be set to lower values for users who have previously achieved high levels of reliability (e.g., Platinum reliability level for over a year).

Below, examples will be considered in which the relative relationship of the reliability score is ascertained with respect to three threshold values. More particularly, examples will be considered in which three threshold values of 10, 30, and 50 have been set. When the reliability score is greater than 50 (RS>50), then a reliability level of “Platinum” may be assigned. When the reliability score is greater than 30 and not more than 50 (30<RS≤50), then a reliability level of “Gold” may be assigned. When the reliability score is greater than 10 and not more than 30 (10<RS≤30), then a reliability level of “Silver” may be assigned. When the reliability score is not more than 10 (RS≤10), then a reliability level of “Bronze” may be assigned.

For example, consider an example for a first patient, Jane, who tests quarterly, and was recently tested 7 days ago. She has a low match count and no recent travel. The reliability score may be computed as follows:

$$RS = ( \frac{30}{7} ) + ( 10 \times 4 ) - ( 5 \times 3 ) - ( 3 \times 5 ) - ( 8 \times 0 ) = 84.29 \qquad Equation 2$$

With a calculated reliability score of 84.29, Jane may be associated with a reliability level with respect to the set of medical data of Platinum. This high reliability level reflects a high commitment to safe dating practices and verified identity.

Consider an additional example for a second patient, Mike. He is a moderate dater who tests every six months and was last tested 30 days ago. He has 8 matches in the predetermined time period, and no recent travel to high risk areas. The reliability score may be computed as follows:

$$RS = ( \frac{30}{30} ) + ( 10 \times 2 ) - ( 5 \times 8 ) - ( 3 \times 6 ) - ( 8 \times 0 ) = 41 \qquad Equation 3$$

Thus, Mike may be associated with a reliability level of Gold. This reliability level reflects responsible testing habits and verified identity.

Consider an additional example for a third patient, Sarah. She tests annually, was last tested three months ago, has a moderate match count, and recently traveled to a low-risk region. The reliability score may be computed as follows:

$$RS = ( \frac{30}{90} ) + ( 10 \times 4 ) - ( 5 \times 3 ) - ( 3 \times 2 ) - ( 8 \times 1 ) = - 11.33 \qquad Equation 4$$

Thus, Sarah may be associated with a reliability level of “Silver.” It reflects her moderate reliability with room for improvement.

Consider an additional example for a fourth patient, Tom. He tests once a year, was last tested six months ago, has a high match count, and recently traveled to a low-risk region. The reliability score may be computed as follows:

$$RS = ( \frac{30}{180} ) + ( 10 \times 1 ) - ( 5 \times 15 ) - ( 3 \times 10 ) - ( 8 \times 1 ) = - 102.83 \qquad Equation 5$$

Thus, Tom may be associated with a reliability level of “Bronze,” in view of the low testing frequency, length of time since the last test, and other factors.

It should be noted that the actual results of the medical testing (e.g., whether a user/patient is diagnosed with an STD or not), or the specific health status of the user (e.g., what type of STD a patient has been diagnosed with) are not used in computation of the reliability score. That is, the computation of the reliability score is independent of the medical test results. In this way, the reliability level can be understood as representing a user's commitment to accountability and safe dating practices and encourage users to receive regular medical testing regardless of the presence or absence of any particular health conditions.

Although examples of computing the reliability score were described above with reference to a specific mathematical formula, it will be understood that the method of calculating the reliability score is not particularly limited herein, and any suitable method can be used to compute the reliability score based on the set of reliability factor data.

Next, at Step S309, the issuing unit 224 may issue a verified digital credential corresponding to the reliability level determined in Step S308 to a set of authorized third parties over the communication network. As described herein, the verified digital credential refers to digital, cryptographically secured information indicating the relative reliability of the first user with respect to the set of medical data. In embodiments, the verified digital credential may be protected using cryptographic techniques such as public-private key infrastructure (PKI) and stored in a secure format such as a digital wallet or the distributed ledger 290.

As an example, in embodiments, the issuing unit 224 may issue the verified digital credential as a PKI digital certificate. More particularly, the issuing unit 224 may generate a public-private key pair or request generation of a public-private key pair from a Certificate Authority. Here, the private key is kept secret and can be used to sign the verified digital credential or encrypt sensitive information (e.g., medical data). The public data is shared publicly and is used to verify signatures or decrypt information encrypted by the private key. The issuing unit 224 may generate a verified digital credential including identification information for a first user, the reliability level (and/or reliability score) calculated for the first user at Step S307, and other relevant metadata (e.g., timestamps, expiration dates, a reference to the corresponding public key), and use the private key to digitally sign the verified digital credential. The signature may be created by hashing the contents of the verified digital credential and encrypting the hash with the private key. As the signature proves the credential was issued by the issuing unit 224, and any changes to the verified digital credential would invalidate the signature, in this way it is possible to maintain integrity and authenticity of the verified digital credential.

The issuing unit 224 may share the signed, verified digital credential with a set of authorized third parties such as the dating service device 280. More particularly, the issuing unit 224 may share the signed, verified digital credential with one or more dating services corresponding to the dating service account information included in the registration information provided by the first user for user account creation. The issuing unit 224 may issue the signed, verified digital credential in association with an identifier that uniquely identifies the specific account on the dating service to which the signed, verified digital credential should be associated. The dating platform associated with the dating service device 280 may validate the verified digital credential by using the public key to decrypt the signature and comparing the decrypted hash to a new hash generated from the credential's contents. If the two hashes match, then the verified digital credential can be confirmed to be authentic and unaltered.

Next, at Step S310, the issuing unit 224 can periodically or continuously update the verified digital credential based on changes to the reliability of the first user. In embodiments, the issuing unit 224 can periodically or continuously monitor for additional medical data for the first user or changes to one or more of the reliability factors included in the set of reliability factor data used to determine the reliability level of the first user in Step S308 and determine an updated reliability level for the first user. More particularly, the issuing unit 224 may collect a second set of reliability factor data from the verified data source, determine a second reliability level of the first user with respect to the set of medical data by evaluating the second set of reliability factor data with respect to the set of reliability conditions, and issue an updated verified digital credential corresponding to the second reliability level to the set of authorized third parties. As the processes for updating the verified digital credential substantially correspond to the processes described above for initially issuing the verified digital credential, a redundant description thereof will be omitted here.

According to the verification management method 300 described above with reference to FIG. 3, it is possible to issue a verified digital credential demonstrating the reliability of a user with respect to a set of medical data while protecting user privacy. This verified digital credential can be used in a dating platform to foster safer and more authentic interactions in online dating environments.

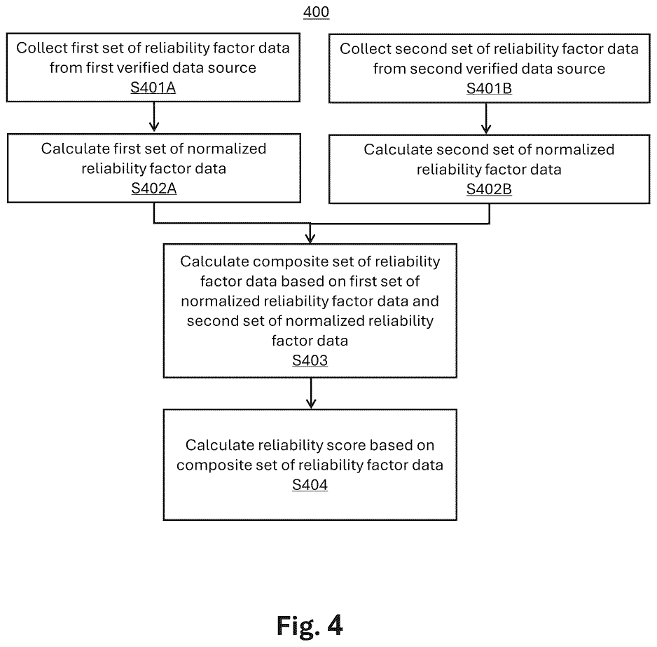

Next, with reference to FIG. 4, an example of a reliability factor aggregation method according to the embodiments of the present disclosure will be described.

Aspects of the present disclosure relate to the recognition that direct combination or comparison of reliability factors collected from different sources, such as different dating services, may be associated with challenges due to underlying differences in the structure, scale, and usage of each respective dating service. For example, a greater number of active users on a first dating service may result in a significantly higher number of matches for a typical user on the first dating service as compared to a second dating service with a lower total number of active users. As another example, a typical user may spend more time engaged with a first dating service that requires users to search for potential matches manually than a second dating service that automatically promotes matches to users. The insight gained via direct combination or comparison of such different metrics may be limited. Accordingly, aspects of the disclosure relate to normalizing reliability factors collected from different sources in order to facilitate meaningful combination thereof and provide greater insight into the reliability of a user as viewed across a plurality of sources.

FIG. 4 is a diagram illustrating an example of the flow of a reliability factor aggregation method 400 according to the embodiments of the present disclosure. The reliability factor aggregation method 400 relates to a method for collecting reliability factor data from a plurality of sources and respectively normalizing the collected reliability factors. The normalized reliable factors may be used to calculate composite reliability factor data that provides a representation of the reliability factor data across multiple sources. The reliability factor aggregation method 400 may be implemented by the components of the verification management system 200 described above with reference to FIG. 2.

First, at Steps S401A and S401B, the collecting unit 222 collects a first set of reliability factor data from a first verified data source and collects a second set of reliability factor data from a second verified data source. In embodiments, collecting the first and second sets of reliability factor data may include querying verified data sources including the medical testing device 260, the dating service device 280, and the distributed ledger 290 for particular information related to the first user identified in Step S304. In embodiments, the first set of reliability factor data and the second set of reliability factor data may be discrete sets of the set of reliability factor data that include one or more reliability factors such as the medical testing frequency that indicates how frequently the first user receives medical testing, the medical testing date that indicates when a medical test was conducted, the number of matches of the first user on one or more dating platforms, the activity level of the first user on one or more dating platforms, or the travel information indicating locations traveled to by the first user. The first verified data source and the second verified data source may be separate verified data sources.

For convenience of explanation, in the following, an example will be described of aggregating reliability factors related to the number of matches and activity level of a first user across a plurality of dating platforms. More particularly, in certain embodiments, the first set of reliability factor data may include a first number of matches of the first user on a first dating service or a first activity level of the first user on the first dating service. The first verified data source may include a first dating service device associated with the first dating service. Similarly, the second set of reliability factor data may include a second number of matches of the first user on the second dating service or a second activity level of the first user on the second dating service. The second verified data source may include a second dating service device associated with the second dating service. Here, the activity level may include one or more of a number of logins to a dating service by the first user in a predetermined time period or an amount of time spent using a dating service by the first user in a predetermined time period. In embodiments, the collecting unit 222 may collect the first number of matches of the first user on the first dating service and/or the first activity level of the first user on the first dating service by querying the first dating service device. Similarly, in embodiments, the collecting unit 222 may collect the second number of matches of the first user on the second dating service and/or the second activity level of the first user on the second dating service by querying the second dating service device.

Next, at Steps S402A and S402B, the collecting unit 222 may calculate a first set of normalized reliability factor data and a second set of normalized reliability factor data, respectively. In embodiments, the collecting unit 222 may calculate the first set of normalized reliability factor data using a first reliability factor baseline obtained from the first verified data source and may calculate the second set of normalized reliability factor data using a second reliability factor baseline obtained from the second verified data source. For example, in the case that the first set of reliability factor data is a first number of matches of the first user on the first dating service, the collecting unit 222 may calculate a first normalized number of matches of the first user on the first dating service based on a first baseline number of matches for users on the first dating service. Similarly, in the case that the second set of reliability factor data is a second number of matches of the first user on the second dating service, the collecting unit 222 may calculate a second normalized number of matches of the first user on the second dating service based on a second baseline number of matches for users on the second dating service. As another example, in the case that the first set of reliability factor data is a first activity level (e.g., number of logins in a predetermined time, usage time) of the first user on the first dating service, the collecting unit 222 may calculate a first normalized activity level of the first user on the first dating service based on a first baseline activity level on the first dating service. Similarly, in the case that the second set of reliability factor data is a second activity level (e.g., number of logins in a predetermined time, usage time) of the first user on the second dating service, the collecting unit 222 may calculate a second normalized activity level of the first user on the second dating service based on a second baseline activity level on the second dating service. In embodiments, the first set of normalized reliability factor data and the second set of normalized reliability factor data may be calculated as follows:

$${R}_{x} = \frac{X - μ}{δ} \qquad Equation 6$$

In Equation 6, Rx is a normalized reliability factor value, X is the raw value of the reliability factor to be normalized (e.g., the number of matches or activity level of a user on a particular dating service), μ is the baseline of the reliability factor to be normalized (e.g., the baseline number of matches or baseline activity level of users on a particular dating service), and σ is the standard deviation of the reliability factor to be normalized (e.g., the standard deviation in the number of matches or activity level of users on a particular dating service). In this way, reliability factor data from different sources can be normalized while accounting for variations in the reliability factors across the different sources.

Next, at Step S403, the collecting unit 222 may calculate a composite set of reliability factor data based on the first set of normalized reliability factor data calculated in Step S402A and the second set of normalized reliability factor data calculated in Step S402B. The composite set of reliability factor data is an aggregated value calculated based on the first set of normalized reliability factor data and the second set of normalized reliability factor data that represents a particular reliability factor as viewed across a plurality of sources. In the case that the first set of normalized reliability factor data is a first normalized number of matches and the second set of normalized reliability factor data is a second normalized number of matches, the collecting unit 222 may calculate a composite number of matches for the first user across the first dating service and the second dating service based on the first normalized number of matches and the second normalized number of matches. Similarly, in the case that the first set of normalized reliability factor data is a first normalized activity level and the second set of normalized reliability factor data is a second normalized activity level, the collecting unit 222 may calculate a composite activity level for the first user across the first dating service and the second dating service based on the first normalized activity level and the second normalized activity level. In embodiments, the composite set of reliability factor data may be calculated as follows:

$${R}_{s} = \frac{{R}_{1} + {R}_{2} + … {R}_{n}}{{N}_{R}} \qquad Equation 7$$

In Equation 7, Rₛ is the composite set of reliability factor data, R₁ is the first set of normalized reliability factor data, R₂ is the second set of normalized reliability factor data, Rₙ is the nth set of normalized reliability factor data, and N_{R} is the number of sets of normalized reliability factor data. As an example of calculating the composite set of reliability factor data for a reliability factor of number of matches, consider a first user who has 20 matches on a first dating service with a baseline of 10 matches/user and a standard deviation of 5, and 60 matches on a second dating service with a baseline of 30 matches/user and a standard deviation of 10. In this case, the first set of normalized reliability factor data R₁ may be calculated to be 2, and the second set of normalized reliability factor data R₂ may be calculated to be 3, such that when divided by the number of sets of normalized reliability factor data N_{R} of 2, the composite set of reliability factor data may be calculated to be 2.5. It will be understood that while examples have been described herein of calculating the composite reliability score based on a first set of normalized reliability factor data and a second set of normalized reliability factor data, the embodiments of the present disclosure are not limited hereto, and the composite reliability score may be calculated based on any number of sets of normalized reliability factor data from any number of verified data sources. Further, it will be understood that the composite reliability factor may be calculated for any one of the reliability factors and is not particularly limited to the number of matches or activity level of the user.

Next, at Step S404, the determining unit 223 may calculate the reliability score based on the composite set of reliability factor data. More particularly, the determining unit 223 may use the value of the composite set of reliability factor data for the value of the corresponding reliability factor in Equation 1 described above to calculate the reliability score.

According to the reliability factor aggregation method 400 described above, it is possible to account for underlying differences in the structure, scale, and usage of different data sources (e.g., dating services) in order to aggregate reliability factors and provide insight into the reliability of a user as viewed across a plurality of different data sources.

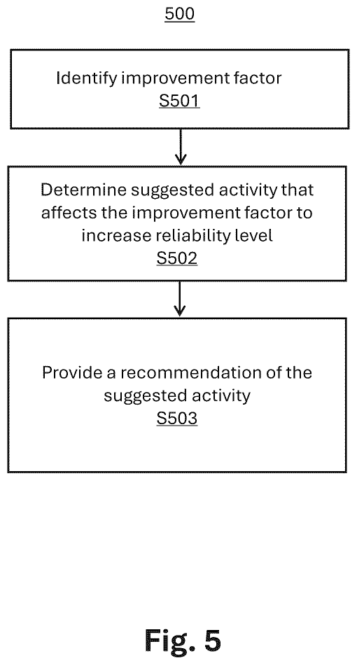

Next, with reference to FIG. 5, an example of an activity suggestion method according to the embodiments of the present disclosure will be described.

Aspects of the disclosure relate to the recognition that in some circumstances, it may be desirable for users to understand how to increase their reliability level. Accordingly, aspects of the disclosure relate to an activity suggestion method 500 for providing recommendations of suggested activities to users that may increase their reliability level. The activity suggestion method 500 may be implemented by the recommending unit 225 of the verification management device 210.

FIG. 5 is a diagram illustrating an example of an activity suggestion method 500 according to the embodiments of the present disclosure. First, at Step S501, the recommending unit 225 may identify an improvement factor based on the set of reliability factor data. As used herein, the improvement factor refers to an aspect, property, or characteristic of the set of reliability factor data that may be modified to effectively increase the reliability level of the first user. In embodiments, the improvement factor may be one of the reliability factors described herein (e.g., the medical testing frequency, the medical testing date, the number of matches, the activity level, or the travel information). In embodiments, the recommending unit 225 may identify the improvement factor by comparing each of the reliability factors to a predetermined improvement factor threshold and identify reliability factors that achieve the threshold as improvement factors.

As an example, in the case that a reliability factor of medical testing frequency achieves an improvement factor threshold of “less than 2 tests per year,” the medical testing frequency may be identified as an improvement factor, as increasing the medical testing frequency can be effective for increasing the reliability score, and therefore the reliability level, of the first user. As an additional example, in the case that a reliability factor of number of matches achieves an improvement factor threshold of “more than 15 matches per week,” the number of matches may be identified as the improvement factor, because decreasing the number of matches can be effective for increasing the reliability score, and therefore the reliability level, of the first user. As an additional example, in the case that a reliability factor of activity level achieves an improvement factor threshold of “more than 10 hours of engagement time per week,” the activity level can be identified as the improvement factor, because decreasing the engagement time can be effective for increasing the reliability score, and therefore the reliability level, of the first user.

Next, at Step S502, the recommending unit 225 may determine a suggested activity that affects the improvement factor identified in Step S501 to increase the reliability level of the first user. As used herein, the suggested activity refers to any action that may be performed by the user to increase their reliability level. In embodiments, the recommending unit 225 may determine the suggested activity using a rule-based approach that maps particular improvement factors to particular suggested activities. As an example, for an improvement factor of medical testing frequency, the recommending unit 225 may reference a look-up table that designates a suggested activity of “increase medical testing frequency” with respect to an improvement factor of medical testing frequency. In certain embodiments, the recommending unit 225 may use a machine-learning technique to analyze the reliability factors of the first user to determine the suggested activity. For example, the recommending unit 225 may use a convolutional neural network or generative artificial intelligence module trained to determine the suggested activity based on the unique reliability factors of the first user.

Next, at Step S503, the recommending unit 225 may provide a recommendation of the suggested activity to the first user. In embodiments, providing the recommendation of the suggested activity to the first user may include transmitting a notification indicating the suggested activity to the user device 250 associated with the first user. In embodiments, providing the recommendation of the suggested activity may include providing an explanation of how performing the suggested activity may impact the reliability level of the first user. For example, the recommendation may provide an explanation such as “increasing your medical testing frequency to 4 times a year will increase your reliability level from Silver to Gold.” In embodiments, the recommendation may include additional information to facilitate performance of the suggested activity. As an example, in the case of a suggested activity of “increase medical testing frequency,” the recommending unit 225 may compare location data collected from the user device 250 (or alternately included in the registration information provided by the first user for account creation) with a database of medical testing facility locations to identify a plurality of candidate medical testing facilities that could provide medical testing services to the first user. As another example, the recommending unit 225 may provide recommendations that take into account the relationship between the reliability factors, such as “If increasing your medical testing frequency is challenging, keeping your number of matches to less than 5 a week will increase your reliability level from Bronze to Silver.”

According to the activity suggestion method 500, users can be provided with recommendations of suggested activities that may increase their reliability level. In this way, it is possible to facilitate transparency with regard to what steps a user can take to increase their reliability level, thereby encouraging user engagement and satisfaction.

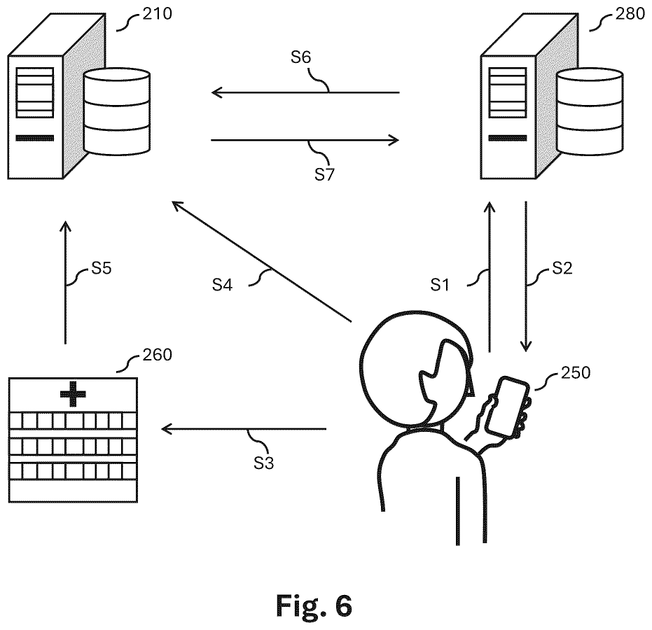

Next, with reference to FIG. 6, a particular example of the flow of the verification management system 200 according to the embodiments of the present disclosure will be described. As illustrated in FIG. 6, a first user may use a user device 250. The user device 250 may be configured to communicate with the verification management device 210, the medical testing facility device 260, and the dating service device 280 over a communication network 270 (not illustrated in FIG. 6).

The first user may use the user device 250 to access a dating service provided by the dating service device 280. For example, the first user may use a mobile application executing on the user device 250 to access a dating platform of the dating service and search for a dating partner. More particularly, as shown in Step S1, the user device 250 may send a request to search for a dating partner, a request to match with a searched partner, or the like. In response to the request sent from the user device 250 in Step S1, in Step S2, search results, a matching result, or the like may be sent from the dating service device 280 to the user device 250. The activity of the first user on the dating platform (e.g., number of logins, engagement time) may be stored in a database of the dating service device 280.

Next, in Step S3, the first user may undergo medical testing at a medical testing facility such as a clinic. The medical testing may include one or more tests to detect the presence or absence of an STD, including but not limited to gonorrhea, chlamydia, syphilis, Human Immunodeficiency Virus (HIV), Human Papillomavirus (HPV), hepatitis B and C, or Herpes Simplex Virus. The medical test results may be stored together with a first authenticated ID for the first user as a set of medical data in a secure database of the medical testing facility device 260.

Next, in Step S4, the first user may use the user device 250 to access a verification service provided by the verification management device 210 in order to verify the set of medical data generated at the medical testing facility in Step S3 for use with one or more dating platforms. The user device 250 may send a service request to the verification management device 210. The service request may include a user account creation request for creating a new account to use the verification service, a request for issuing a verified digital credential to authenticate the set of medical data, or both. The service request may include a second authenticated ID for the first user to prove the identity of the first user.

In response to receiving the service request sent by the user device 250, the verification management device 210 performs processing according to the embodiments of the present disclosure as described above with reference to FIGS. 2-5. That is, in Step S5, the verification management device 210 may receive the set of medical data from the medical testing facility device 260, verify the identity of the user based on the first and second authenticated IDs, and in Step S6, the verification management device 210 may collect a set of reliability factor data from one or more verified data sources such as the dating service device 280. Additionally, in Step S6, the verification management device 210 can determine the reliability level of the set of medical data based on the collected set of reliability factor data. Next, in Step S7, the verification management device 210 may issue and transmit a verified digital credential to an authorized third party such as the dating service device 280. The dating service device 2603 may use the verified digital credential to represent the reliability of the first user on an associated dating platform. For example, the verified digital credential may be represented as a badge in the profile of the first user indicating that medical test results for the first user have been independently verified. The badge may be displayed with a visual indicator indicating the reliability of the first user (e.g., Platinum, Gold, Silver, Bronze) to provide an indication to other users of the dating platform of the degree of reliability of the first user.

As described herein, aspects of the verification management technique according to the embodiments of the present disclosure relate to issuing a verified digital credential demonstrating the reliability of a user with respect to a set of medical data. This verified digital credential can be used in a dating platform to provide an indication that the set of medical data associated with a particular user has been independently verified.

By issuing a verified digital credential for use on a dating platform that indicates that medical data and identity documents for a particular user have been independently verified, it is possible to authenticate real users and reduce the prevalence of fake accounts on dating platforms. As a result, it is possible to mitigate the technical challenges posed by fake accounts, such as the need to employ computationally expensive monitoring techniques, and thereby reduce the amount of computing resources needed for fake account detection. Similarly, the amount of computing resources (e.g., memory, processing resources, storage space) spent maintaining fake accounts can be reduced. Additionally, by reducing the prevalence of fake accounts it is possible to mitigate the risk of data breaches and facilitate greater security of user's personal data.

Aspects of the present disclosure may be associated with a variety of benefits for partnering medical facilities, dating platforms, and their end users. For example, by providing a verification management technique that awards higher reliability levels to users who undergo more frequent medical testing, medical facilities may benefit from increased testing demand. Additionally, providing a verified digital credential that can be used in a dating platform to demonstrate the reliability of a user may foster a safe, authentic dating environment that reduces the likelihood of users encountering fake accounts or catfishers, facilitating the creation of genuine connections between responsible users who have demonstrated a commitment to health and accountability. Further, providing a verified digital credential that can be used in a dating platform to demonstrate the reliability of a user may be beneficial for the dating platform as more users choose to use the dating platform due to its safety and security.

In this way, it becomes possible to reduce the prevalence of fraudulent profiles, catfishing, and deceptive behaviors while fostering safer and more authentic interactions in online dating environments, and simultaneously mitigate technical challenges related to computer resource management for fake account prevention.

### Definitions

The following includes definitions of selected terms employed herein. The definitions include various examples or forms of components that fall within the scope of a term and that may be used for implementation. The examples are not intended to be limiting. Both singular and plural forms of terms may be within the definitions.

An “operable connection,” or a connection by which entities are “operably connected,” is one in which signals, physical communications, or logical communications may be sent or received. Typically, an operable connection includes a physical interface, an electrical interface, or a data interface, but it is to be noted that an operable connection may include differing combinations of these or other types of connections sufficient to allow operable control. For example, two entities can be operably connected by being able to communicate signals to each other directly or through one or more intermediate entities like a processor, operating system, a logic, software, or other entity. Logical or physical communication channels can be used to create an operable connection.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and so on, have been illustrated by describing examples, and while the examples have been described in considerable detail, it is not the intention of the applicants to restrict or in any way limit scope to such detail. It is, of course, not possible to describe every conceivable combination of components or methodologies for purposes of describing the systems, methods, and so on, described herein. Additional advantages and modifications will readily appear to those skilled in the art. Therefore, the invention is not limited to the specific details, the representative apparatus, and illustrative examples shown and described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

What is claimed is:

1. A verification management device comprising:
a processor; and
a memory connected to the processor and including a computer readable program,
wherein:
the verification management device is communicably connected, via a communication network, to at least a medical testing facility device associated with a medical testing facility, and
the computer readable program, when executed on the processor, causes the processor to:
receive, from the medical testing facility device via the communication network, a set of medical data characterizing a health status of a first user;
collect, from a verified data source over the communication network, a set of reliability factor data for evaluating reliability of the first user with respect to the set of medical data;
determine, by evaluating the set of reliability factor data with respect to a set of reliability conditions, a reliability level of the first user with respect to the set of medical data; and
issue, to a set of authorized third parties over the communication network, a verified digital credential corresponding to the reliability level.

2. The verification management device according to claim 1, wherein determining the reliability level of the first user with respect to the set of medical data further comprises:
calculating, based on the set of reliability factor data, a reliability score for the first user;
determining, by comparing the reliability score with a first threshold value; a relative relationship between the reliability score and the first threshold value; and
assigning, from among a plurality of candidate reliability levels, the reliability level to the set of medical data based on the relative relationship between the reliability score and the first threshold value.

3. The verification management device according to claim 2, wherein the set of reliability factor data includes one or more reliability factors selected from the group consisting of:
a medical testing frequency that indicates how frequently the first user receives medical testing;
a medical testing date that indicates when a medical test was conducted;
a number of matches of the first user on one or more dating platforms;
an activity level of the first user on one or more dating platforms; and
travel information indicating locations traveled to by the first user.

4. The verification management device according to claim 3, wherein calculating, based on the set of reliability factor data, the reliability score for the first user further comprises:
assigning a weighting value to each reliability factor of the set of reliability factor data; and
computing the reliability score using a mathematical formula that defines a relationship between the one or more reliability factors and the weighting values assigned to each reliability factor.

5. The verification management device according to claim 3, wherein:
the verification management device is communicably connected via the communication network to a set of dating service devices each associated with a different dating service; and
the verified data source includes one or more of a user device associated with the first user, the medical testing facility device, the dating service device, and a distributed ledger.

6. The verification management device according to claim 5, wherein the set of authorized third parties includes the set of dating service devices.

7. The verification management device according to claim 5, wherein collecting, from a verified data source over the communication network, the set of reliability factor data for evaluating reliability of the first user with respect to the set of medical data further comprises:
collecting, from a first dating service device of the set of dating service devices that is associated with a first dating service, a first number of matches of the first user on the first dating service;
calculating, based on the first number of matches of the first user on the first dating service and a first baseline number of matches for users on the first dating service, a first normalized number of matches of the first user on the first dating service;
collecting, from a second dating service device of the set of dating service devices that is associated with a second dating service, a second number of matches of the first user on the second dating service;
calculating, based on the second number of matches of the first user on the second dating service and a second baseline number of matches for users on the second dating service, a second normalized number of matches of the first user on the second dating service; and
calculating, based on the first normalized number of matches and the second normalized number of matches, a composite number of matches for the first user across the first dating service and the second dating service for use in calculating the reliability score for the first user with respect to the set of medical data.

8. The verification management device according to claim 5, wherein collecting, from a verified data source over the communication network, the set of reliability factor data for evaluating reliability of the first user with respect to the set of medical data further comprises:
collecting, from a first dating service device of the set of dating service devices that is associated with a first dating service, a first activity level of the first user on the first dating service;
calculating, based on the first activity level of the first user on the first dating service and a first baseline activity level for users on the first dating service, a first normalized activity level of the first user on the first dating service;
collecting, from a second dating service device of the set of dating service devices that is associated with a second dating service, a second activity level of the first user on the second dating service;
calculating, based on the second activity level of the first user on the second dating service and a second baseline activity level for users on the second dating service, a second normalized activity level of the first user on the second dating service; and
calculating, based on the first normalized activity level and the second normalized activity level, a composite activity level for the first user across the first dating service and the second dating service for use in calculating the reliability score for the first user with respect to the set of medical data.

9. The verification management device according to claim 8, wherein the activity level includes one or more of a number of logins to a dating service by the first user in a predetermined time period or an amount of time spent using a dating service by the first user in a predetermined time period.

10. The verification management device according to claim 1, wherein the set of medical data indicates test results of medical testing of the first user for sexually transmitted diseases.

11. The verification management device according to claim 1, wherein the processor is further configured to:
collect, from the verified data source, a second set of reliability factor data;
determine, by evaluating the second set of reliability factor data with respect to the set of reliability conditions, a second reliability level of the first user with respect to the set of medical data; and
issue, to the set of authorized third parties, an updated verified digital credential corresponding to the second reliability level.

12. The verification management device according to claim 1, wherein the processor is further configured to:
provide a recommendation to the first user for increasing the reliability level.

13. The verification management device according to claim 12, wherein providing a recommendation to the first user for increasing the reliability level further includes:
identifying, based on the set of reliability factor data, at least one improvement factor for improving the reliability level of the first user;
determining at least one suggested activity which affects the at least one improvement factor to increase the reliability level; and
providing a recommendation of the at least one suggested activity to the first user.

14. The verification management device according to claim 1, wherein the set of medical data is encrypted by the medical testing facility device and transmitted through a secure channel of the communication network, the set of medical data is associated with a first authenticated ID for a first patient, and the processor is further configured to:
receive, from a user device via the communication network, a user account creation request associated with a second authenticated ID of the first user;
create a user account for the first user based on the user account creation request;
decrypt the set of medical data received from the medical testing facility device;
verify an identify of the first user by comparing the first authenticated ID associated with the set of medical data with the second authenticated ID associated with the user account creation request; and
associate, in response to determining that the first authenticated ID associated with the medical data matches the second authenticated ID associated with the user account creation request, the set of medical data with the user account.

15. The verification management device according to claim 1, wherein the verified digital credential is issued to the set of authorized third parties using Public Key Infrastructure.

16. The verification management device according to claim 15, wherein issuing, to a set of authorized third parties over the communication network, the verified digital credential corresponding to the reliability level further comprises:
generating a key pair including a public key and a private key;
generating the verified digital credential based at least on an authenticated ID for the first user and the reliability level determined for the first user;
digitally signing the verified digital credential by generating a hash corresponding to the verified digital credential and encrypting the hash using the private key; and
transmitting the verified digital credential to the set of authorized third parties for validation using the public key.

17. The verification management device according to claim 16, wherein issuing, to a set of authorized third parties over the communication network, the verified digital credential corresponding to the reliability level further comprises:
recording the verified digital credential to a distributed ledger.

18. The verification management device according to claim 1, wherein receiving, from the medical testing facility device via the communication network, the set of medical data characterizing a health status of the first user further comprises:
retrieving, using a transaction identifier associated with the first user, the set of medical data from a distributed ledger to which the set of medical data has been recorded by the medical testing facility device.

19. A verification management system comprising:
a verification management device;
a user device associated with a first user;
a distributed ledger; and
a medical testing facility device associated with a medical testing facility, wherein the verification management device, the user device, the distributed ledger and the medical testing facility device are communicably connected via a communication network,
the verification management device including:
a processor; and
a memory connected to the processor and including a computer readable program that when executed by the processor, causes the processor to:
receive, from the user device via the communication network, a user account creation request associated with user registration information, the user registration information including a first authenticated ID for the first user;
receive, from the medical testing facility device via the communication network, a set of medical data indicating medical test results for a first patient, the set of medical data associated with a second authenticated ID for the first patient;
create a user account for the first user based on the user account creation request;
verify, by comparing the second authenticated ID associated with the set of medical data for the first patient with the first authenticated ID included in the user registration information for the first user, that the first patient corresponds to the first user;
collect, from a verified data source over the communication network, a set of reliability factor data for evaluating reliability of the first user with respect to the set of medical data;
store, in the distributed ledger, the set of medical data and the set of reliability factor data in association with the first authenticated ID for the first user;
calculate, based on the set of reliability factor data, a reliability score for the first user;
determine, by comparing the reliability score with a first threshold value; a relative relationship between the reliability score and the first threshold value;
assign, from among a plurality of candidate reliability levels, the reliability level to the set of medical data based on the relative relationship between the reliability score and the first threshold value; and
issue, to a set of authorized third parties over the communication network, a verified digital credential corresponding to the reliability level.

20. A verification management method for a verification management device, the verification management device including:
a processor; and
a memory connected to the processor and including a computer readable program,
wherein:
the verification management device is communicably connected, via a communication network, to at least a medical testing facility device associated with a medical testing facility, and
the computer readable program, when executed on the processor, causes the processor to perform a verification management method comprising:
receiving, from the medical testing facility device via the communication network, a set of medical data characterizing a health status of a first user;
collecting, from a verified data source over the communication network, a set of reliability factor data for evaluating reliability of the first user with respect to the set of medical data;
determining, by evaluating the set of reliability factor data with respect to a set of reliability conditions, a reliability level of the first user with respect to the set of medical data; and
issuing, to a set of authorized third parties over the communication network, a verified digital credential corresponding to the reliability level.
